(19) United States
(12) Patent Application Publication
(10) Pub. No.: US 2014/0243825 A1
(43) Pub. Date: Aug. 28, 2014

(54) COMPRESSIBLE DEVICE ASSEMBLY AND ASSOCIATED METHOD FOR FACILITATING HEALING BETWEEN BONES

(71) Applicant: MYLAD ORTHOPEDIC SOLUTIONS LLC, Arlington, VA (US)

(72) Inventors: Ronald Arthur Yapp, Manchester, MI (US); Scott G. Edwards, Arlington, VA (US); Paul Stuart Cooper, Potomac, MD (US)

(73) Assignee: MYLAD ORTHOPEDIC SOLUTIONS LLC, Arlington, VA (US)

(21) Appl. No.: 14/168,610

(22) Filed: Jan. 30, 2014

Related U.S. Application Data
(63) Continuation of application No. 13/029,897, filed on Feb. 17, 2011, now abandoned.

(51) Int. Cl. A61B 17/66

(52) U.S. Cl. CPC A61B 17/66
USPC 606/57

(57) ABSTRACT

A method of applying compression between a distal bone portion and a proximal bone portion using an orthopedic device is provided. Methods may include inserting the orthopedic device through the proximal bone portion and into the distal bone portion, where the orthopedic device includes a core and a sleeve. The method may include fastening the core to the distal bone portion and fastening the sleeve to the proximal bone portion. The method may include applying compression between the distal bone portion and the proximal bone portion. The method may include maintaining compression between the distal bone portion and the proximal bone portion with a pawl member. Methods may also include allowing for dynamic compression between the distal bone portion and the proximal bone portion after the initial compression has been achieved and maintaining the dynamic compression between the distal bone portion and the proximal bone portion with the pawl member.

### CROSS-REFERENCE TO RELATED APPLICATIONS

This application claims priority to U.S. patent application Ser. No. 13/029,897 filed on Feb. 17, 2011, and published as U.S. Patent Application Publication No. 2012/0215222, and is herein incorporated by reference in its entirety.

### FIELD

Orthopedic devices for treating arthritic joints or repairing bone defects and, more specifically, to orthopedic device assemblies for providing fusion, fixation, compression, and/or stabilization of the ankle (tibiotalar) and subtalar (talocalcaneal) joints.

### BACKGROUND

Orthopedic devices, such as nails, rods, or pins, are often used in the medical field for fusing bones or bone segments across a joint to correct deformities, treat arthritis, or remedy other issues with procedures such as with a tibiotalocalcaneal arthrodesis. Such orthopedic devices may also be used to treat fractures of long bones, such as in the humerus, radius, ulna, tibia, fibula, femur, metacarpal, and metatarsal, or other non-long bones, such as the calanceus and other tarsal or carpal bones. Such devices are typically designed to be inserted across a joint or fracture site into the bone on either side of the joint or fracture, and generally are fastened to the bones on either side of a joint or to bone segments on either side of the fracture to stabilize the bone and promote proper fusion or healing.

In some cases, the bones or bone segments on either side of a joint or fracture are spaced apart and must be brought closer together to promote fusion or healing. Devices have been proposed that provide compression between bones or bone segments by fixing the orthopedic device to one bone (or bone segment) and then moving the second bone towards the bone in which the device is fixed by way of an external device which applies compression to the end of the second bone. The second bone is then secured to the orthopedic device and the joint is allowed to fuse or the fracture is allowed to heal. However, these compression providing devices must be securely and removably attached to the orthopedic device while not compromising the integrity of the orthopedic device or the ability of the compression device to provide appropriate compression. Further, existing compression mechanisms may apply compression across weak bones or by pressing on the surface of a patient's skin, both of which may result in negative complications. In some cases, a drill guide must also be securely and removably attached to the orthopedic device.

Thus, there remains a need for an orthopedic device assembly that is easy to install without the need for extensive surgical dissection, and provides appropriate compression of the bone to promote fusion or healing.

### BRIEF SUMMARY

Example embodiments of the present invention generally related to an orthopedic device for providing compression across a joint, fracture, or defect of a bone. One example embodiment of an orthopedic device may include a core with at least one opening configured to receive a fastener for securing the core to a first bone portion, and at least one ratchet or pawl member. The orthopedic device may further include a sleeve including at least one open end for slidably receiving the core, at least one opening configured to receive a fastener for fastening the sleeve to a second bone portion, and at least one ratchet or pawl member disposed at least partially within the sleeve and configured to engage the ratchet or pawl member of the core. The engaged ratchet and pawl member may cooperate to allow the core to move into the sleeve in a direction through the open end and preclude the core from moving out of the sleeve in the opposite direction.

The pawl member may include at least one pawl surface that is biased against a ratchet surface of the ratchet. The core may include a slot where the slot is configured to receive at least a portion of the pawl member. The sleeve may include an inner bore into which the core is received where the inner bore includes the ratchet surface. The orthopedic device may further include an end cap configured to limit the distance the core can advance into the sleeve. The orthopedic device may further include at least one fastener insert disposed within the at least one opening of the sleeve, where the at least one fastener insert is configured to engage the fastener received through the at least one opening of the sleeve. The core may include at least one slot where the at least one fastener insert may be configured to pass through the at least one slot of the core and the at least one fastener insert may be configured to preclude relative rotation between the core and the sleeve.

Another example of an orthopedic device assembly according to embodiments of the present invention may include an orthopedic device including a core and a sleeve. The assembly may further include a target guide and a compression assembly configured to attach the orthopedic device to the target guide, where the compression assembly may be configured to draw the core into the sleeve. The orthopedic device may further include a pawl member disposed between the core and the sleeve where the pawl member is configured to allow the core to be drawn into the sleeve and preclude the core from sliding out of the sleeve. The compression assembly may include a core attachment bolt configured to engage the core and draw the core into the sleeve in response to the core attachment bolt turning with respect to the core. The orthopedic device may include at least one hole disposed in the core configured to receive a fastener and at least one hole disposed in the sleeve configured to receive a fastener. The fastener received in the at least one hole disposed in the core may be configured to attach the core to a distal bone portion and a fastener received in the at least one hole disposed in the sleeve may be configured to attach the sleeve to a proximal bone portion. The at least one hole disposed in the sleeve may be configured to receive at least one fastener insert, where the fastener received through the at least one hole in the sleeve engages the at least one fastener insert. The core may further include at least one slot where the at least one fastener insert may be configured to pass through the at least one slot of the core and preclude relative motion between the sleeve and the core. Compression may be applied between the distal bone portion and the proximal bone portion in response to the compression assembly drawing the core into the sleeve.

Example embodiments of the present invention may provide a method of applying compression between a distal bone portion and a proximal bone portion using an orthopedic device. The method may include inserting the orthopedic device through the proximal bone portion and into the distal bone portion, where the orthopedic device includes a core and a sleeve. The method may further include fastening the core to the distal bone portion and fastening the sleeve to the proximal bone portion. The method may further include applying initial compression between the distal bone portion and the proximal bone portion. The method may further include maintaining compression between the distal bone portion and the proximal bone portion with a pawl member. The method may also include allowing for dynamic compression between the distal bone portion and the proximal bone portion after the initial compression has been achieved and maintaining the dynamic compression between the distal bone portion and the proximal bone portion with the pawl member. The method may still further include limiting the dynamic compression between the distal bone portion and the proximal bone portion. Applying initial compression between the distal bone portion and the proximal bone portion may include drawing the core into the sleeve. Drawing the core into the sleeve may include using a compression assembly to draw the core into the sleeve. Using a compression assembly to draw the core into the sleeve may include engaging a threaded bore of the core with a threaded stud and rotating the threaded stud relative to the core. The core may be held in rotational alignment with the sleeve.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 is a perspective view of an assembled orthopedic device according to an example embodiment of the present invention;

FIG. 2 is an exploded perspective view of an orthopedic device according to an example embodiment of the present invention;

FIG. 3 is a section view of several components of an orthopedic device according to an example embodiment of the present invention;

FIG. 4 is a section view of the components of FIG. 2B as assembled according to an example embodiment of the present invention;

FIG. 4A is a section view of the components of an assembled orthopedic device according to another example embodiment of the present invention;

FIG. 5 is a perspective view of a target guide according to an example embodiment of the present invention;

FIG. 6 is a perspective view of a target guide with a patient attachment mechanism according to an example embodiment of the present invention;

FIG. 7 is an illustration of an orthopedic device, target guide, and attachment mechanism as inserted and attached to a patient;

FIG. 8 is a perspective view of a target guide, compression assembly, orthopedic device, and attachment mechanism according to an example embodiment of the present invention;

FIG. 9 is an illustration of a compression assembly according to an example embodiment of the present invention;

FIG. 10 depicts a compression assembly as secured to a target guide according to an example embodiment of the present invention;

FIG. 11 illustrates a compression assembly as secured to a target guide and attached to an orthopedic device according to an example embodiment of the present invention;

FIG. 12 illustrates the target guide of FIG. 11, further comprising a leverage bridge according to an example embodiment of the present invention;

FIG. 13 is a perspective view of a target guide together with a screw guide and drill guide according to an example embodiment of the present invention;

FIG. 14 is an illustration of a screw guide and a drill guide according to an example embodiment of the present invention;

FIG. 15 illustrates a screw guide insert as aligned within the target guide according to an example embodiment of the present invention;

FIG. 16 illustrates a screw guide insert, screw guide, and drill guide according to an example embodiment of the present invention;

FIG. 17 illustrates an appendage as located within a target guide with the orthopedic device inserted into the boneand the drill guide insert in contact with the bone according to an example embodiment of the present invention; and

FIG. 18 depicts an orthopedic device and several compression limiting caps of varying lengths according to an example embodiment of the present invention.

### DETAILED DESCRIPTION OF THE DRAWINGS

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Embodiments of the present invention generally relate to an orthopedic device assembly for fusing bones, bone segments, or bone portions in order to fuse joints, repair fractures, repair bone defects, or otherwise add rigidity to a bone or bone portions. For ease of explanation, however, the specification and accompanying figures will refer to fusion of a joint, and in particular, the ankle (tibiotalar) or subtalar (talocalcaneal) joint, although it is to be understood that any type of bone repair or fusion, including the repair of fractures, osteotomies, and other bone defects or fusion sites, and combinations thereof, may be accomplished using embodiments of the device described herein. Further, while example embodiments of the present invention are illustrated as fusing two bones together, example embodiments may be used to join two or more different bones together for fusion, join two or more bone segments together to heal across a fracture, or otherwise strengthen a single or multiple bones. As such, the term “bone portion” is used herein to describe any portion of a bone up to and including an entire bone such that fusion of a first bone portion with a second bone portion may describe two separate bones or two portions of a single bone. Further, while example embodiments of the present device describe the fusion of two bone portions or bone segments, example embodiments may further apply compression between more than two bone segments, such as, for example, applying compression between the tibia, talus, and calcaneus bones. Compression applied and maintained by example embodiments of the present invention may be applied across any number of bones or bone segments wherein the compression is applied coaxially across the bones or bone segments. The illustrated example embodiments in which compression is applied across two bones is provided for illustration and is not intended to be limiting.

As described further below, the orthopedic device assembly includes an orthopedic device that may be configured to be inserted through the calcaneus bone of the foot, through the talus, and into the tibia. An orthopedic device according to the present invention may be inserted through the calcaneus bone of the foot, through the talus, and into the core of the tibia bone such that the orthopedic device is disposed at least partially within the tibia, partially within the talus, and partially within the calcaneus. The orthopedic device assembly may include a compression assembly that may be configured to facilitate compression between the calcaneus and the talus, and the talus and the tibia. By fastening the installed orthopedic device to the tibia on a distal side of the joint and to the calcaneus on the proximal side of the joint and subsequently compressing the orthopedic device, the calcaneus bone and the tibia may be drawn towards one another such that compression is applied across the joint to facilitate fusion. The orthopedic device may further be installed through the talus bone disposed between the tibia and calcaneus wherein the talus bone may be secured to the orthopedic device or the talus may be secured by compression applied on either side of the talus between the tibia and calcaneus bones. Once the orthopedic device is installed, the patient may or may not be able to use the affected appendage during the fusion process. In other example embodiments wherein the orthopedic device is configured for the repair of a fracture, the fracture may be entirely across the bone creating two separate bone portions or may only extend partially across the bone creating two partially separate bone portions. The orthopedic device may be inserted along the axis of the fractured bone, such as along the intramedullary canal of a long bone, and extend across the fracture. The orthopedic device may provide compression between the two bone portions across the fracture to facilitate healing of the fracture.

In this regard, the terms “proximal” and “distal” refer to locations relative to the insertion site of the assembly after it has been inserted into the bone rather than anatomical convention. In other words according to example embodiments provided herein, the proximal bone may refer to the calcaneus bone where the orthopedic device is inserted while the distal bone may refer to the tibia that receives a distal portion of the orthopedic device assembly.

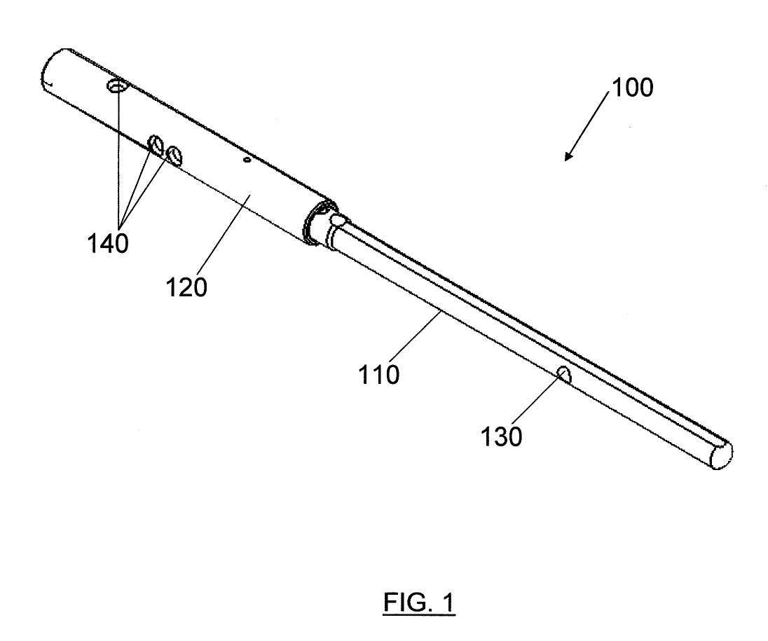

FIG. 1 illustrates an orthopedic device 100 according to an example embodiment of the present invention. The orthopedic device 100 comprises two primary portions: the core 110 and the sleeve 120. The core is slidingly received within the sleeve to permit axial movement between the two portions. The core 110 may include one or more attachment holes 130 or slots (not shown) for receiving a fastener in order to secure the core 110 within a distal bone portion. The sleeve 120 may also include one or more attachment holes 140 or slots configured to receive a fastener for securing the sleeve within the proximal bone portion. The attachment holes 130, 140 may include threaded bores or smooth bores depending upon the fastening mechanism as will be further described below.

The orthopedic device 100 may be used in conjunction with a target guide to perform an arthrodesis, as described below. The orthopedic device 100 may be configured (i.e., shaped and sized) to be inserted into a bone and fastened to the bone on either side of the joint. Thus, the particular configuration of the orthopedic device 100 may vary depending on the type and size of the bone and joint to be treated. Furthermore, the device may be made of any absorbable or non-absorbable material that is compatible for use inside the human body, such as titanium, stainless steel, cobalt chrome, plastic, carbon fiber, or polymer. The core 110 and sleeve 120 may, for example, be made of a rigid material such as titanium alloy which may provide the necessary strength and rigidity while being substantially non-reactive with the human body.

In the embodiment illustrated in FIG. 1, for example, the orthopedic device 100 is configured for use in an adult tibia. However, the orthopedic device 100 and assembly described below may be used in various other locations in the human body, such as for repairing fractures of the femur, humerus, ulan, radius, fibula, lateral malleolus (distal fibula) at the ankle, metacarpal, metatarsal, or calcaneus or other bones of the carpus, or fusing joints such as the tibitalar joint, talocalcaneal joint, joints of the midfoot, knee, or wrist. The core 110 of the orthopedic device of FIG. 1 is tapered, with the proximal end (i.e., the end closest to the insertion point when installed) having a slightly larger diameter than the distal end (i.e., the end farthest from the insertion point when installed). Also, the orthopedic device may be tapered in the reversed manner or remain uniform in diameter throughout its length. The axis of the orthopedic device 100 may be straight, as shown in FIG. 1, or curved.

The orthopedic device 100 may include a number of holes 130, 140 configured to receive fasteners for fastening bones, or portions thereof, to the orthopedic device 100. One or more of the holes 130 may be located towards the distal end of the orthopedic device 100, for example to fasten a bone, such as a tibia that is on a distal side of the joint, to the orthopedic device 100, whereas one or more other holes 140 may be located towards the proximal end of the orthopedic device 100, for fastening another bone portion, such as the calcaneus, that is on a proximal side of the joint, as discussed below. Furthermore, the holes 130, 140 may be configured to receive various types of fasteners, such as pins, bolts, pegs, screws, and locking screws, among others. In some cases, the holes 130, 140 may be internally-threaded to receive corresponding externally threaded fasteners. The holes 130, 140 may have a chamfered opening on the side configured to receive a corresponding fastener which may aid insertion of the fastener by providing a larger opening to accept and guide the fastener.

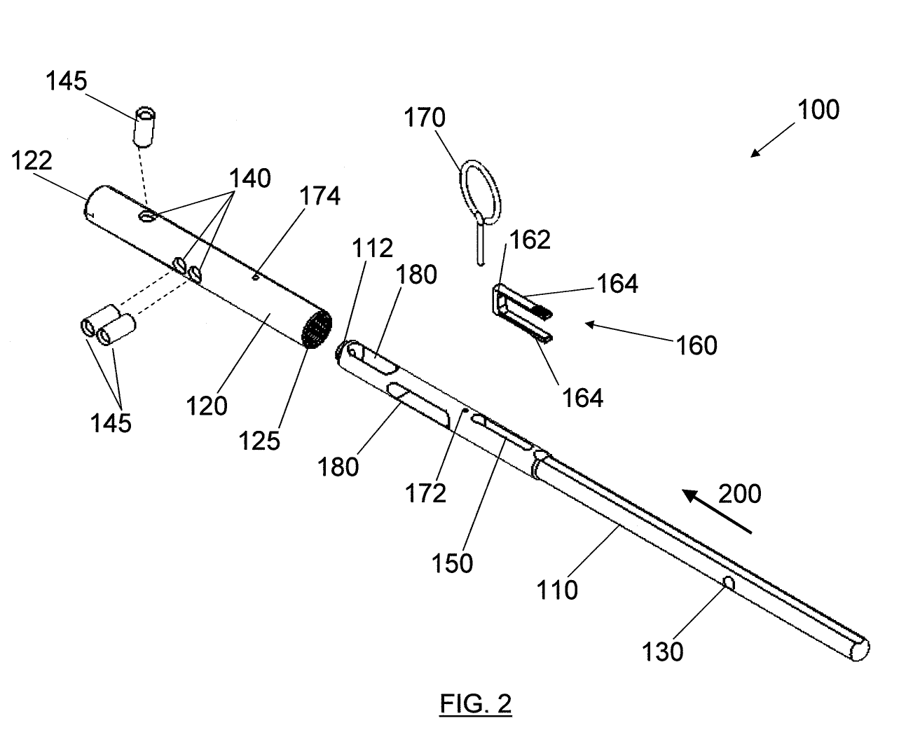

FIG. 2 illustrates the orthopedic device of the example of FIG. 1 in an exploded view with the core 110 separated from the sleeve 120. The core 110 may include a slot 150 configured to receive a pawl member 160. The depicted pawl member 160 includes a transverse portion 162 and two lateral portions 164 which are resiliently biased outward. The pawl member 160 may be made of a material that permits limited flexibility while retaining strength and durability, such as a stainless steel or titanium alloy. The outwardly biased lateral portions each include a toothed pawl surface disposed on the outward face which are configured to engage a complementary ratchet surface 125 disposed on the inside bore of the sleeve 120. The pawl surface and ratchet surface cooperate to permit motion of the core 110 in a first direction into the bore of the sleeve 120 (i.e., in the direction of arrow 200) and prevent relative motion between the core 110 and the sleeve 120 in a second direction, opposite the first direction, out of the sleeve 120.

Any number of ratcheting mechanisms may be employed beyond what is illustrated in the figures as will be apparent to one of ordinary skill in the art. However, one particular advantage to the illustrated pawl member 160 is that the ratchet surfaces are not outwardly exposed when the core 110 and sleeve 120 are assembled. Enclosing the pawl and ratchet surfaces within the orthopedic device 100 may be advantageous because the ratchet surfaces will not become obstructed with tissue or bone and the orthopedic device maintains a relatively smooth external surface which aids installation and is better for maintaining sterility. When the core 110 is assembled with the sleeve 120, a pin 170 may be configured to be inserted through hole 174 of the sleeve 120 and through hole 172 of the core 110 to prevent relative motion between the core 110 and the sleeve 120 until the pin 170 is removed prior to the installation of the orthopedic device 100 into a bone. The term “pawl member” is used herein to describe the component engaging the ratchet surface 125 disposed on the inside bore of the sleeve 120 and may describe any member that engages the ratchet surface 125 to allow movement in a first direction and preclude movement in a second, opposite direction. While the depicted embodiment illustrates the pawl member 160 disposed within or proximate the core 110 and the ratchet surface 125 disposed within the sleeve 120, optionally, the ratchet surface may be disposed on the core 110 with a pawl member disposed within the sleeve 120.

The sleeve 120 further comprises fastener inserts 145, which are secured within the holes 140. The fastener inserts 145 may include a threaded bore for receiving a fastener when installed in a bone or they may have a smooth bore through which a fastener may be inserted during attachment to the bone. In the instance of a threaded bore fastener insert 145, the threads may include a locking feature such as a locking-profile thread, a deformable locking member (e.g. an elastic stop nut or elliptical offset locknut type thread) or possibly an adhesive.

The nail is assembled with the pawl member 160 inserted into slot 150 of the core 110. The core 110 is then inserted into the sleeve 120 until hole 172 of the core is aligned with hole 174 of the sleeve at which point the pin 170 may be installed to prevent relative motion between the core 110 and the sleeve 120. The fastener inserts 145 may then be aligned and inserted into holes 140. The fastener inserts 145 may be received through slots 180 of the core 110. The fastener inserts 145 may then be securely attached (e.g. with welding, adhesive, etc.) within the holes 140. The slots 180 permit the core 110 to slide within the sleeve 120 and the fastener inserts 145 both preclude entry of foreign substances (tissue, bone, etc.) into the bore of the sleeve 120 and provide a bore configured to receive a fastener to secure the sleeve 120 to the bone of a patient. The slots 180 further allow the proximal end 112 of the core 110 to be accessible through the proximal end 122 of the sleeve 120 as will be described further below. In addition, the fastener inserts 145 which pass through the slots 180 in the core 110 preclude relative rotation between the core 110 and the sleeve 120. Precluding relative rotation between the core 110 and the sleeve 120 maintains the relative alignment between the bores of the fastener inserts 145 and the fastener holes 130 of the core 110 which may be required to facilitate the drilling of holes and insertion of fasteners as will be described further below.

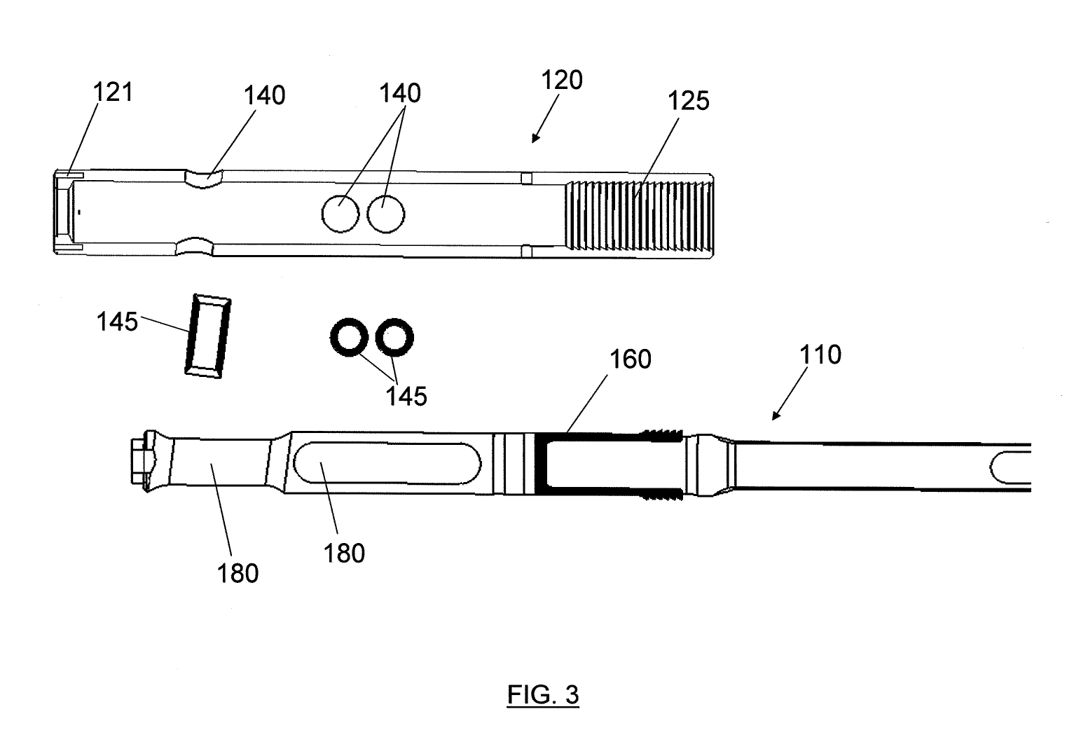

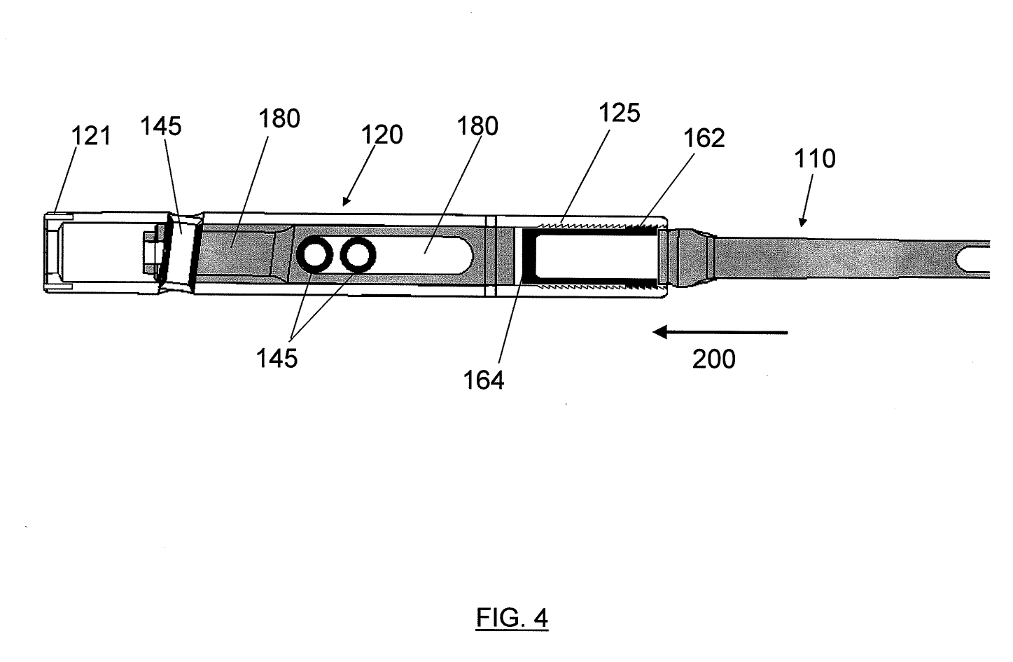

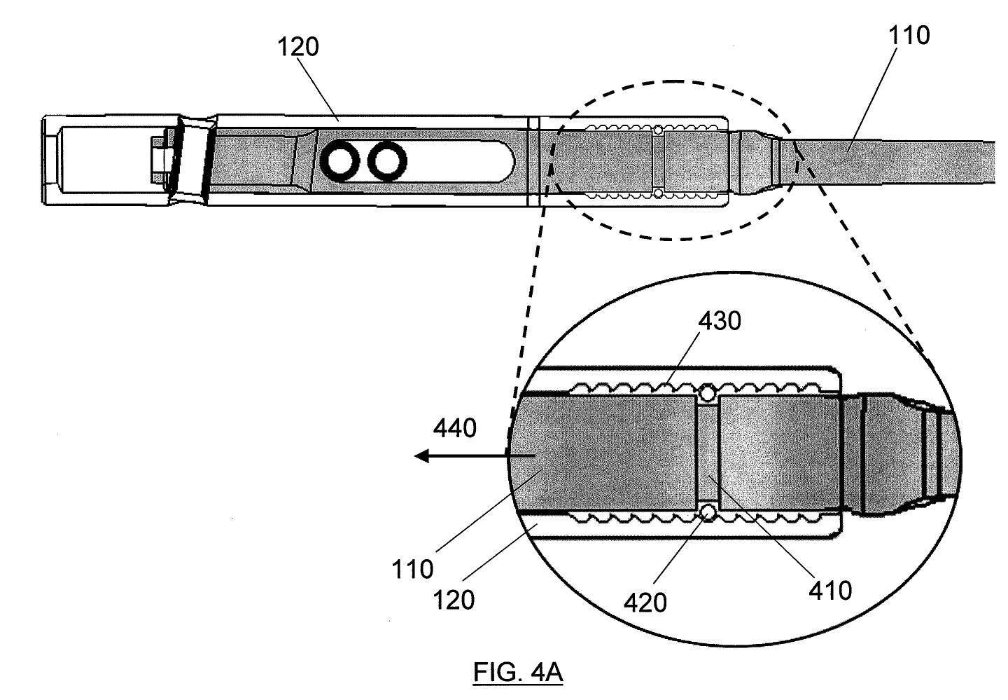

FIG. 3 illustrates a section view of several components of the orthopedic device 100 of FIG. 2 depicting the core 110, the sleeve 120, and the fastener inserts 145. The core 110 includes the pawl member 160 and slots 180. The sleeve 120 includes the internal ratchet surface 125 and the holes 140 together with alignment recesses 121 which will be discussed below. During assembly, with the pawl member 160 inserted into the core 110 as shown, the core 110 is inserted into the bore of the sleeve 120 until the pawl member 160 engages the internal ratchet surface 125 of the sleeve. FIG. 4 illustrates a section view of the core 110 assembled into the sleeve 120. The fastener inserts 145 may then be inserted through holes 140 of the sleeve 120 and through the slots 180 of the core 110. The fastener inserts 145 are secured in place in the holes 140 of the sleeve 120 as described above. The pawl member 160, in cooperation with the internal ratchet surface 125, permits movement of the core 110 into the sleeve 120 in the direction of arrow 200 and precludes movement of the core 110 out of the sleeve 120 in the direction opposite arrow 200. The pawl member 160 engages the internal ratchet surface 125 by virtue of the lateral portions 164, which include a pawl surface, being resiliently biased against the internal ratchet surface 125.

FIG. 4A illustrates another ratchet mechanism according to an example embodiment of the present invention, where the core 110 includes a groove 410 about which is disposed a snap-ring 420. The snap-ring 420 may be configured to have an external diameter which is compressible by virtue of a void in the circumference of the snap-ring 420, allowing the snap ring to be reduced in diameter when a force is applied around the outer surface of the snap-ring 420. The groove 410 may be configured to hold the snap-ring 420 in place on the core 110 while permitting the diameter of the snap-ring 420 to be reduced when sufficient force is applied. The sleeve 120 may include a ratchet surface 430 configured to engage the snap ring 420. The ratchet surface 430 may include a surface with a profile that applies an external force around the diameter of the snap-ring 420 when the core 110 is moved in a first direction (arrow 440). The external force applied to the snap ring 420 as the core 110 is moved in the direction of arrow 440 may cause the diameter of the snap-ring 420 to be reduced thereby allowing the snap-ring 420 to pass from one groove of the ratchet surface 430 to the next groove of the ratchet surface 430 as the core 110 is received within the sleeve 120. The surface profile of the ratchet surface 430 may further be configured to not apply an external force around the diameter of the snap-ring 420 when the core 110 is moved in a direction opposite of arrow 440. As movement of the core 110 in the direction opposite that of arrow 440 does not provide for an eternal force applied around the diameter of the snap-ring 420, the snap-ring 420 will not be able to advance to the next groove within the ratchet surface 430 and motion of the core 110 in the direction opposite of arrow 440 will be precluded. Optionally, the ratchet surface 430 may be designed to permit motion of the core in the direction of arrow 440 with the application of a force in the direction of arrow 440 above a first magnitude and permit motion of the core opposite the direction of arrow 440 with the application of a force opposite the direction of arrow 440 above a second magnitude, that is greater than the first magnitude. The shape of the ratchet surface 430 profile may be designed to dictate the force required in either the direction of arrow 440 or opposite arrow 440 to collapse the core 110 into the sleeve 120 or distract the core 110 from the sleeve 120, respectively.

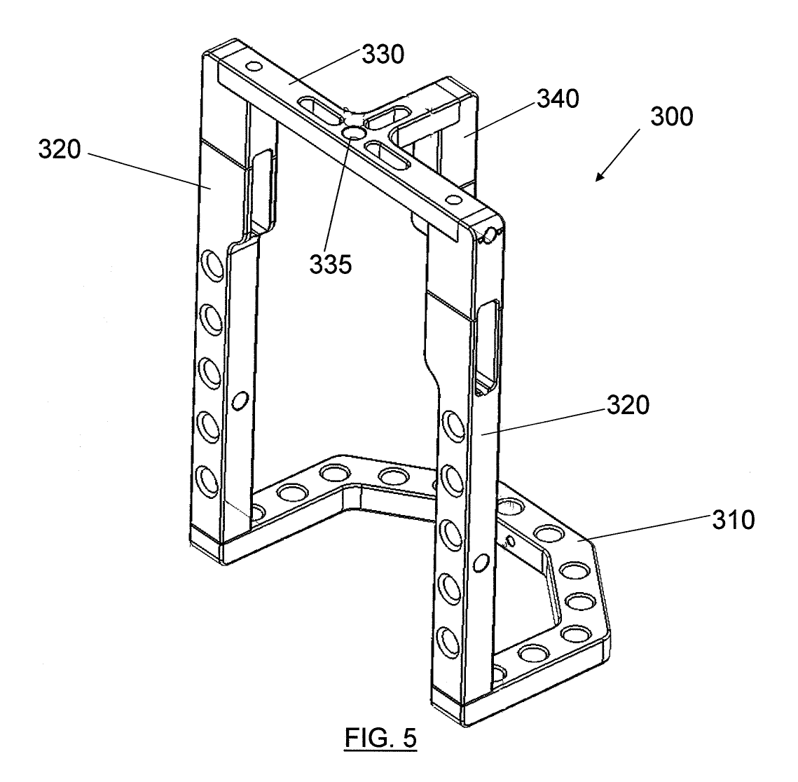

FIG. 5 depicts a target guide according an example embodiment of the present invention. The target guide 300 may be constructed of a radiolucent material to provide a relatively unobstructed view of an attached orthopedic device as inserted into a bone during an X-ray. The target guide may be made of a single piece of material or may be several pieces joined together through any conventional method such as adhesives, epoxies, fasteners, etc. The illustrated embodiment of the target guide 300 includes a base 310, two side pylons 320, a top 330, and a posterior pylon 340. The top 330 includes an attachment hole 335 for attachment of the orthopedic device as will be described further below.

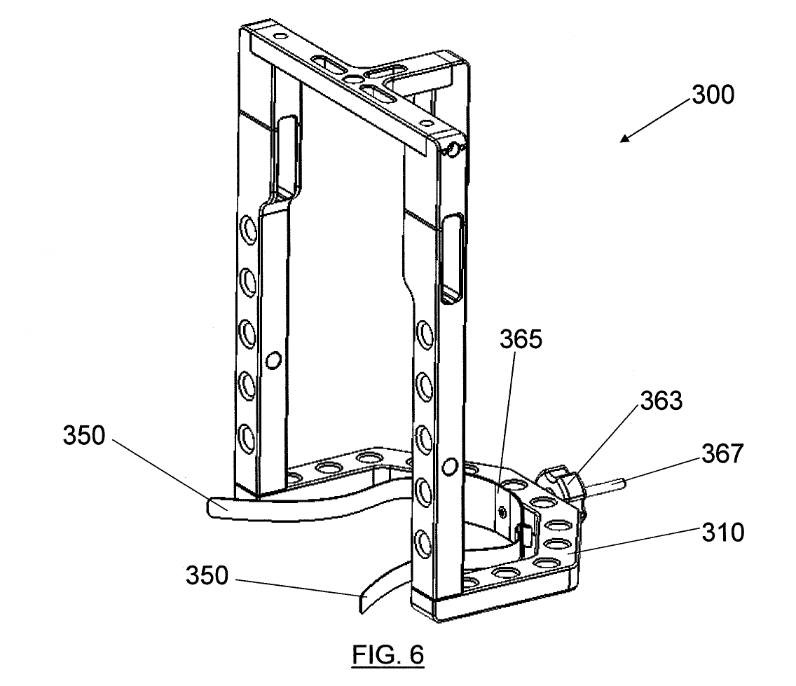

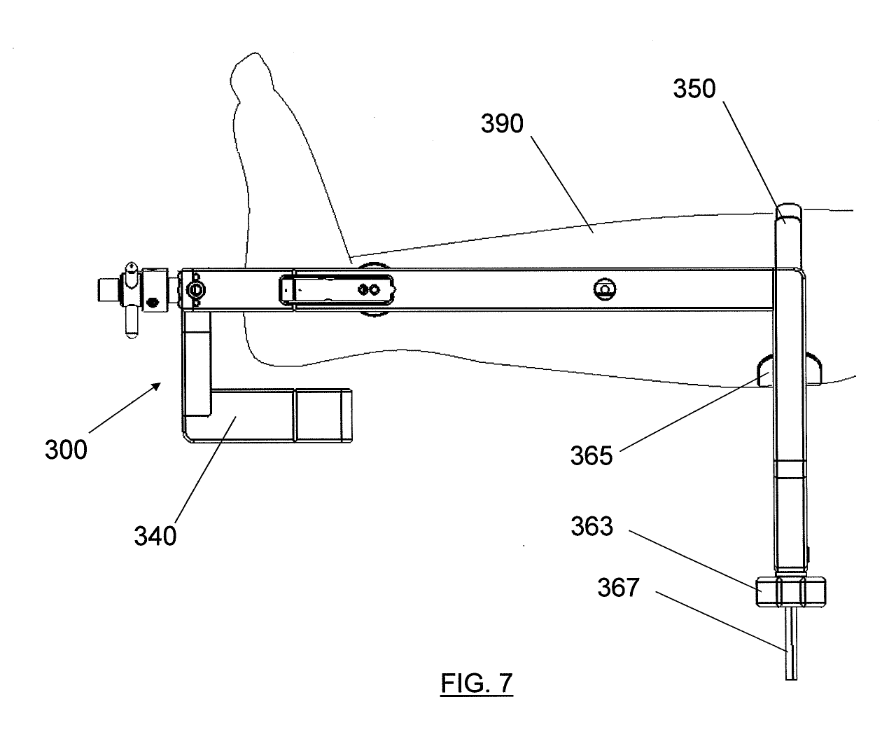

As illustrated in FIG. 6, the target guide 300 may include a mechanism for securing the base 310 to the appendage of the patient, for example around a leg of a patient. The mechanism may include straps 350, such as Velcro straps or a belt, and a positioning device which may help in locating the patient's appendage within the target guide. The positioning device may include a locating pad 365 which may be attached to a threaded stud 367 that passes through the handle 363 of the positioning device. When the handle 363 is turned, the stud 367 may extend or retract as appropriate for locating the appendage within the base 310 of the target guide 300. For example, if the target guide 300 and the orthopedic device are configured to be used for a tibiocalcaneous arthrodesis, as shown in FIG. 7, the straps 350 may be secured around a patient's leg 390 or calf region. The handle 363 may be turned to drive the stud 367 to the appropriate length for locating the locating pad 365 at the appendage 390 of the patient when the appendage 390 is properly positioned within the target guide 300.

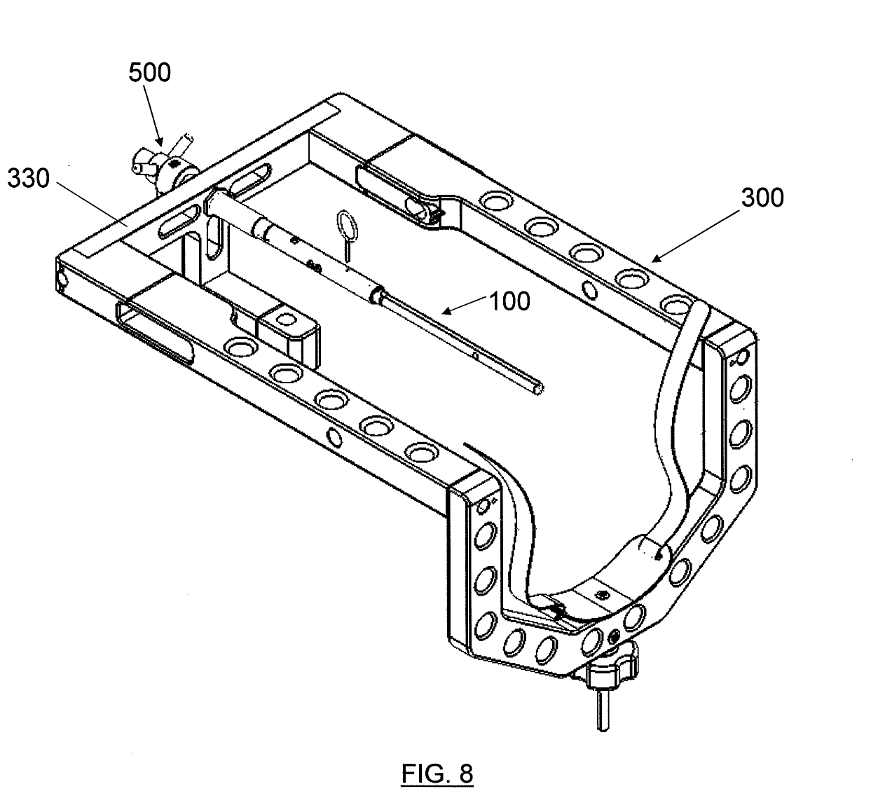

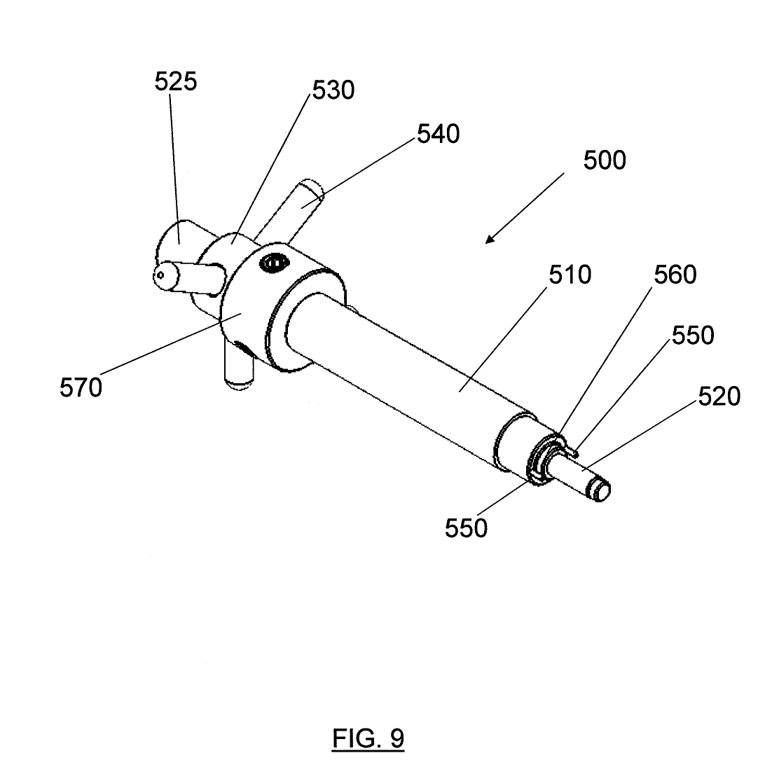

FIG. 8 illustrates a target guide 300 as illustrated in FIGS. 5-7 with an orthopedic device 100 assembled thereto. The orthopedic device 100 is attached to the target guide 300 by a compression assembly 500 that engages the hole 335 in the top 330 of the target guide 300 as shown in FIG. 5. The compression assembly 500 is shown in greater detail in FIG. 9 which depicts the compression assembly 500 as separated from the target guide 300 and the orthopedic device 100. The compression assembly 500 may be attached to the target guide 300 through a number of possible mechanisms including a press fit or locking screws that secure the device compression assembly 500 through the top 330. Optionally, the compression assembly 500 may include a threaded exterior to which could either thread into a threaded bore of hole 335 in the top 330 or pass through a smooth bore and include a locking nut on the compression assembly 500 both above the top 330 and below the top 330, thereby securing the compression assembly 500 to the target guide 300.

The compression assembly 500 includes a compressor sleeve 510 which is held fixed to the top 330 through hole 335. A sleeve attachment bolt includes a top portion 570 at the proximal end of the compression assembly 500 and a shaft 560 that extends through the compressor sleeve 510 and exits the compressor sleeve 510 at the distal end. The shaft includes an externally threaded end 560 which extends beyond the compressor sleeve 510. The externally threaded end 560 may engage an internally threaded proximal end of the sleeve 120 to attach the sleeve 120 to the compressor sleeve 510. The compressor sleeve 510 may further include pins 550 that extend from the distal end of the compressor sleeve 510. The pins 550 may engage corresponding alignment recesses 121 in the proximal end of the sleeve 120 as shown in FIGS. 3 and 4. During attachment of the compression assembly 500 to the orthopedic device 100, the alignment recesses 121 of the sleeve 120 may be brought into alignment with the pins 550 of the compressor sleeve 510. The externally threaded end 560 of the sleeve attachment bolt may engage corresponding internal threads of the sleeve 120. As the top portion 570 of the sleeve attachment bolt is turned, the sleeve attachment bolt 560 draws the sleeve 120 into engagement with the compressor sleeve 510. The pins 550 prevent rotational movement of the sleeve 120 as the sleeve attachment bolt 570 is turned and therefore ensure proper rotational alignment for the fastener holes 130 of the core.

The compression assembly 500 further includes a core attachment bolt 520 that extends through the compressor sleeve 510 and through the sleeve attachment bolt 560, 570. The core attachment bolt is free to rotate within the sleeve attachment bolt 560, 570 independent of the compressor sleeve 510 or the sleeve attachment bolt 560, 570. The core attachment bolt 520 may include a head 525 and a handle, such as turnstile handle 530. The turnstile handle 530 includes spokes 540 configured to permit a user to apply a rotational force to the core attachment bolt 520. The core attachment bolt head 525 may include a hexagonal external shape, an Allen-keyway or Torx® keyway to enable a user to apply torque or for application of a torque wrench or torque-limiting driver to the core attachment bolt 520. The distal end of the core attachment bolt 520 may include an externally threaded portion configured to engage the proximal end of the core 110.

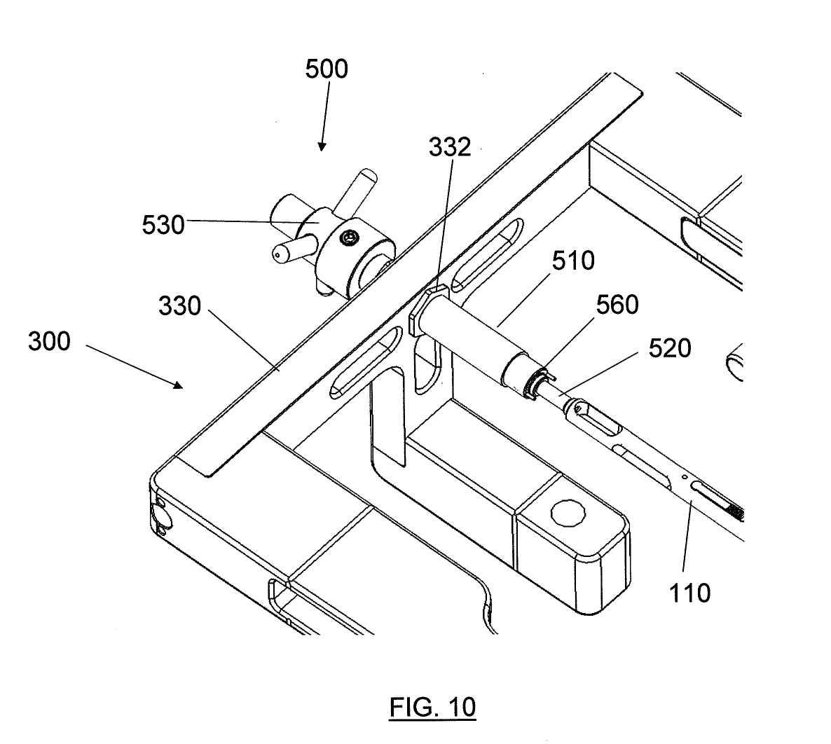

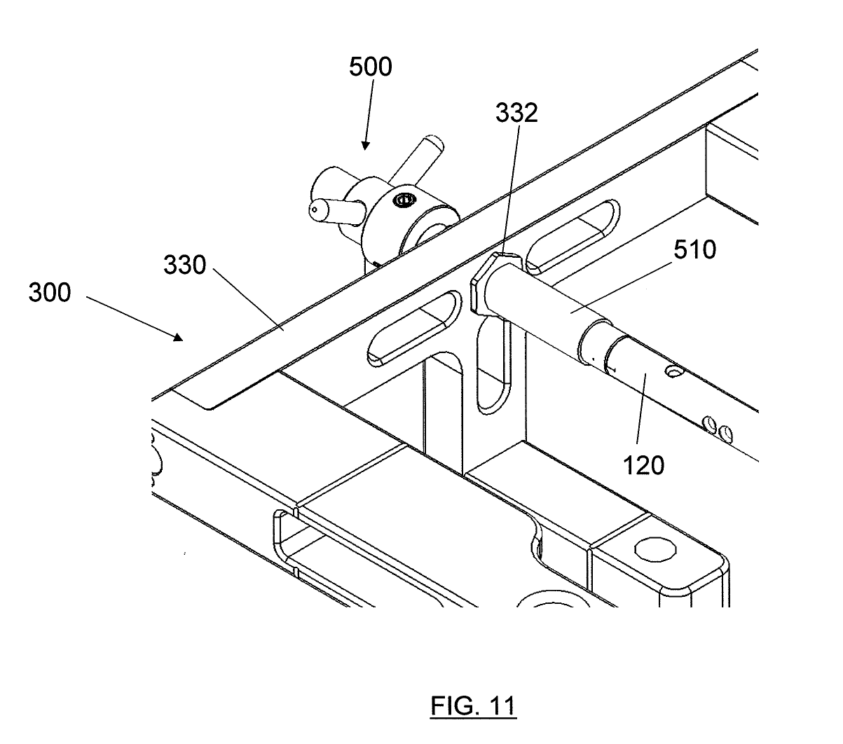

FIG. 10 depicts a target guide 300 with a compression assembly 500 secured to the core 110 of an orthopedic device according to an example embodiment of the invention. The sleeve 120 has been omitted from this illustration for clarity. As illustrated, the compression assembly 500 is secured within the top 330 of the target guide 300 with an attachment nut 332. The core attachment bolt 520 is disposed within the sleeve attachment bolt 560 and compressor sleeve 510, and is attached at a distal end to the proximal end of the orthopedic device core 110. Attachment of the core attachment bolt 520 to the core 110 may be performed by turning the turnstile handle 530 of the compression assembly 500 to engage the threads of the core attachment bolt 520 with the threads of the core 110. FIG. 11 illustrates the target guide 300 of FIG. 10 with the sleeve 120 shown.

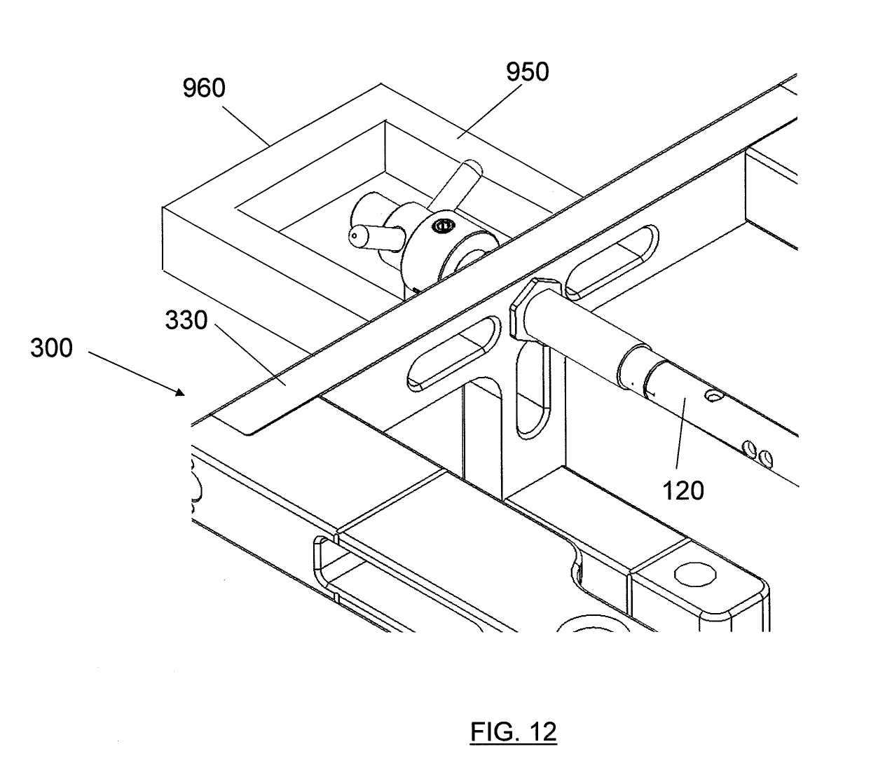

Optionally, the target guide 300 may be configured to receive or engage a leverage bridge 950 as illustrated in FIG. 12. The leverage bridge 950 may span across the compression assembly 500 and provide a surface 960 that is axially aligned with the orthopedic device 100 to which a surgeon may apply a force, such as with a mallet or through manual force, to drive the orthopedic device into the bone or medullar canal of the bone in which the orthopedic device is being inserted.

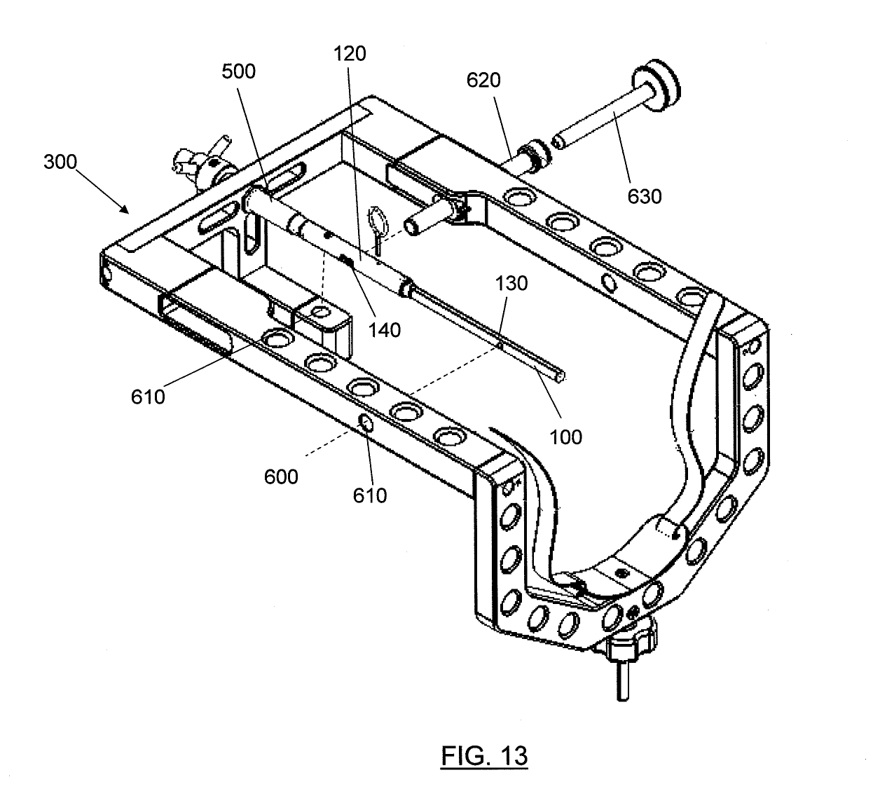

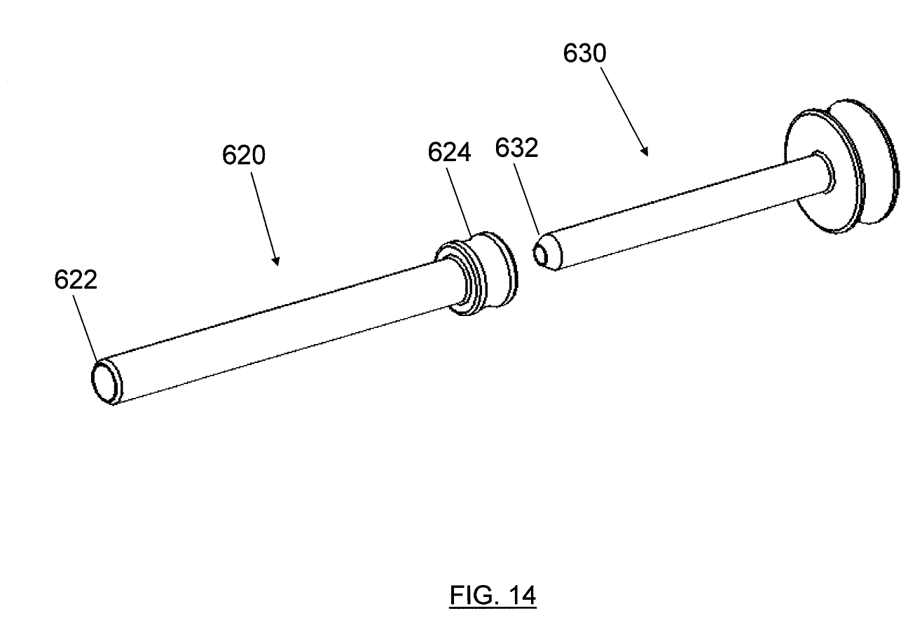

FIG. 13 illustrates a target guide 300 with a compression assembly 500 secured to an orthopedic device that includes a sleeve 120 and a core 110. As illustrated through the dashed lines 600, alignment holes 610 in the target guide 300 correspond to the fastener holes 130, 140 in the orthopedic device core 110 and sleeve 120. Upon insertion of the orthopedic device into the bone of a patient, the fastener holes 130, 140 are no longer visible such that the alignment holes 610 are necessary to guide drill and screw insertion. To that end, a screw guide 620 is provided that can be inserted into the alignment holes 610. FIG. 14 illustrates the screw guide 620 and drill guide 630 in greater detail. The screw guide 620 may include a hollow tube with a distal end 622 having a beveled edge. As the distal end 622 of the screw guide 620 is intended to be inserted through the alignment holes 610, through the flesh, and into contact with the bone that is to be repaired, the beveled distal end 622 of the screw guide 620 may facilitate both better engagement with the bone surface and it may reduce trauma to the surrounding flesh as it is inserted. The screw guide may further include a proximal end 624 that includes a grasping region to aid insertion and removal of the screw guide 620 from the target guide 300 and the patient. A drill guide 630 may be configured for insertion into the hollow bore of the screw guide 620. The drill guide may define a hollow tube with a smaller diameter hole extending therethrough than exists in the screw guide 620. Further, the drill guide 630 may also include a beveled distal end 632 configured to cooperate with the beveled distal end 622 of the screw guide 620 to form a substantially continuous or smooth bevel from the distal tip of the drill guide to the outer diameter of the screw guide 630.

In practice, with an orthopedic device inserted into the bone of a patient and the target guide attached thereto, the drill guide 630 may be inserted into the screw guide 620, and the two may together be inserted through an alignment hole 610 of the target guide 300, through an incision in the skin of the patient, through the flesh, and into contact with the bone. A drill bit attached to a drill may be inserted through the bore of the drill guide 630 to drill a hole through the bone. The drill guide 630 properly locates the position of the drill bit such that a hole created by the drill bit penetrates the bone in alignment with a fastener hole 130, 140 of the orthopedic device. Once the hole has been drilled, the drill guide 630 may be removed from the screw guide 620. As the drill guide 630 occupied the bore of the screw guide 620, the bore of the screw guide 620 may then be substantially free of tissue, bone, or other substances such that a surgeon may insert a screw or other fastener into the bore of the screw guide 620. The screw guide 620 guides the fastener into alignment with a fastener hole 130, 140 of the orthopedic device. A tool, such as a screw driver, Torx® driver, or other tool may then be used to fasten the fastener through the hole drilled in the bone, across the fastener hole 130, 140 of the orthopedic device, and into the bone on the opposite side from the screw guide 620. Once the fastener is inserted and properly tightened, the screw guide 620 may be removed from the alignment hole 610 and the patient.

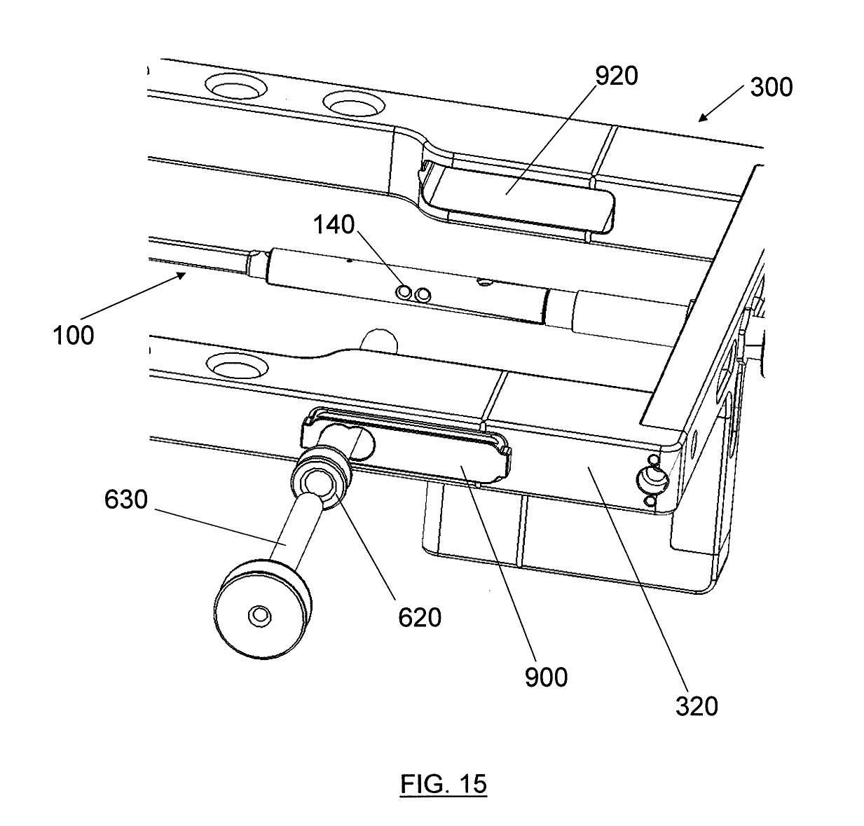

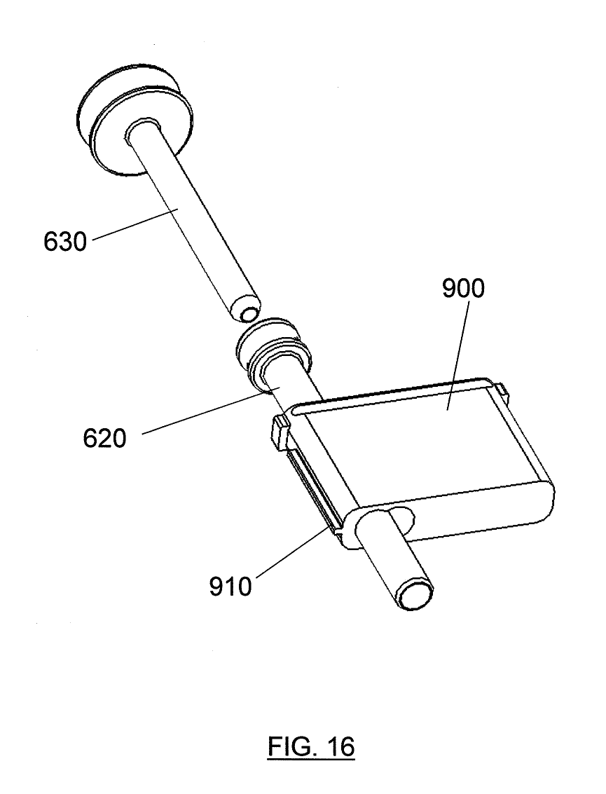

FIG. 15 illustrates an example embodiment of a drill guide 630 and screw guide 620 engaging a screw guide insert 900. The screw guide insert 900 may be made of a radiolucent or non-radiolucent material and is configured to receive the screw guide 620 in a bore to properly align the drilling of a hole and insertion of a fastener in alignment with the fastener holes 140 of an orthopedic device 100. The screw guide insert 900 may be removable from the side pylon 320 of the target guide 300 such that it can be inserted in either side pylon at the option of the user for the optimum drilling and fastener insertion side of the patient's appendage. The removability/replaceability of the screw guide insert 900 permits an opening 920 to be formed in each side pylon 320 that may facilitate a clear view of the orthopedic device from a side-view during an X-ray or provide access to a patient's flesh for preparing an incision into which the drill guide and screw guide may be inserted. As illustrated in FIG. 16, the screw guide insert 900 may include a keyway 910 that prevents improper installation of the screw guide insert 900 into the target guide.

As outlined above, example embodiments of the present invention may provide a method for fusing together bone portions on either side of a joint. Initially, an assembly comprising an orthopedic device 100, a compression assembly 500, and a target guide 300 appropriate for the bone containing the defect may be selected. In the case of an ankle arthrodesis, a hole may be drilled through the calcaneus bone to receive the orthopedic device 100. The bone which is to receive the orthopedic device, such as the tibia, may, in some cases, be prepared beforehand for receiving the orthopedic device 100 using tools and methods known by those skilled in the art, such as by drilling and/or reaming the bone so that the dimensions of the channel formed in the bone correspond to the dimensions of the orthopedic device 100. The orthopedic device may then be inserted through the calcaneus and into the prepared channel of the tibia. For example, referring to FIG. 7, the orthopedic device may be inserted through the calcaneus proximate the heel region of the foot and into the tibia. The orthopedic device may also be configured so that it cuts its own path into the bone with or without the assistance of accessory tools. The assembly may be rotated to achieve the best location for fastening the orthopedic device 100 within the bone based upon the arthrodesis size or shape, the surrounding bone or tissue, or the surgeon's preference. The assembly may then be secured to the patient by use of the mechanism described above with respect to FIG. 7.

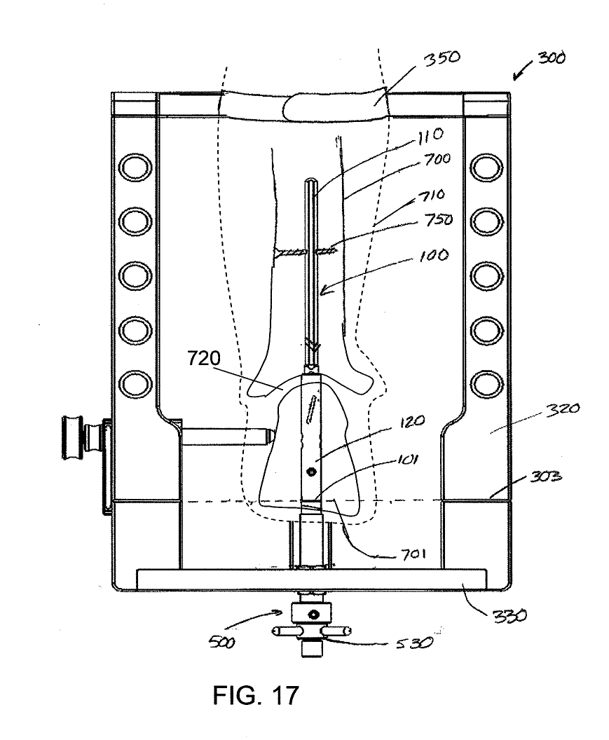

FIG. 17 depicts an assembly comprising an orthopedic device 100 attached to a target guide 300 with a compression assembly 500 according to an example embodiment of the invention. The orthopedic device 100 is illustrated as inserted through the calcaneus bone 701 into the tibia 700 of a patient and the target guide 300 is secured to the patient's appendage 710 with the attachment straps 350. Of note, the pin 170, which advantageously prevents undesired, premature movement between the core 110 and the sleeve 120, must be removed prior to insertion of the orthopedic device 100 into the bone. This arrangement of the pin 170 prevents inadvertent placement of the orthopedic device 100 in the bone while the core and sleeve are still locked to each other.

The calcaneus 701 and tibia 700 and appendage profile of the flesh 710 are shown for illustrative purposes only. Between the calcaneus bone 701 and the tibia 700 is the ankle joint 720. The orthopedic device 100 extends from the calcaneus bone 701, across the joint 720, into the tibia bone 700. As illustrated, the drill guide 630 and screw guide 620 are engaged with the calcaneus bone 701 in position to facilitate a drilling operation as described above. Fastener 750 is illustrated in the installed position wherein the fastener 750 couples together the orthopedic device core 110 and the tibia 700. Additional fasteners may be used depending upon the size of the bone, the fastener size, and the strength of the fastener-bone interface (e.g., a weak bone may require more fasteners to distribute the forces within the bone). Fasteners are also installed through the calcaneus bone 701 and through the sleeve 120 of the orthopedic device 100.

Once the orthopedic device 100 is secured within the bone (i.e., the core 110 is secured to the tibia 700 and the sleeve 120 is secured to the calcaneus bone 701), the screw guide(s) are removed from the target guide 300. The turnstile handle 530 may be turned to draw the core 110 into the sleeve 120, thereby shortening the overall length of the orthopedic device and applying compression across the joint 720. As the turnstile handle 530 is turned, the core attachment bolt 520 draws the core 110 into the sleeve 120. As the core 110 is drawn into the sleeve 120, the pawl member 160 and ratchet surface 125 cooperate to allow movement of the core 110 in the proximal direction into the sleeve 120, but preclude movement of the core 110 in the distal direction, out of the sleeve 120. Thus, as the core 110 advances into the sleeve 120 compression across the joint 720 is achieved and maintained. The length in which the core 110 can be drawn into the sleeve 120 may be configured according to the size and application of the orthopedic device, and may be up to around 15 millimeters for an application such as a tibiotalocalcaneal arthrodesis.

In any case, the target guide 300 and compression assembly 500 may not be needed once the desired amount of compression has been achieved and the orthopedic device 100 compressed to the desired force or distance. As a result, the compression assembly 500 may be disconnected from the orthopedic device by disengagement of the core attachment bolt 520 from the core 110 and the sleeve attachment bolt 560 may be disengaged from the sleeve 120. The attachment straps 350 of the target guide 300 may then be removed from the patient and the target guide 300, together with the compression assembly 500 may be removed. In this way, the orthopedic device 100 may remain in the bone, with the calcaneus 701 and tibia 700 attached to facilitate stabilization of the joint and promote proper fusion and to provide a relatively unobstructed surface of the bone and allow the patient to use the affected part to the extent possible with greater comfort. The orthopedic device 100 may provide compression across both the ankle and subtalar joints in applications such as with a tibiotalocalcaneal arthrodesis.

The proximal end of the orthopedic device 101 is preferably situated such that it does not protrude from the cortex of the calcaneus bone 701 as a protrusion from beneath the heel of a patient could be both uncomfortable and detrimental to the fusion of the joint. The radiolucent target guide 300 may include a groove 303, such as a v-shaped groove, extending at least partially across the side pylons 320 and/or the posterior pylon (not shown in FIG. 17). The groove 303 may be visible both to the naked eye and in an X-ray of the radiolucent target guide 300. The groove 303 is disposed at the same distance from the top portion 330 of the target guide 300 as the proximal end 101 of the orthopedic device 100, which enables a surgeon to determine the location of the proximal end 101 of the orthopedic device 100 relative to the calcaneus bone 701 cortex when the orthopedic device 100 is inserted into a bone 701 and an X-ray image does not clearly depict the proximal end 101 once surrounded by bone.

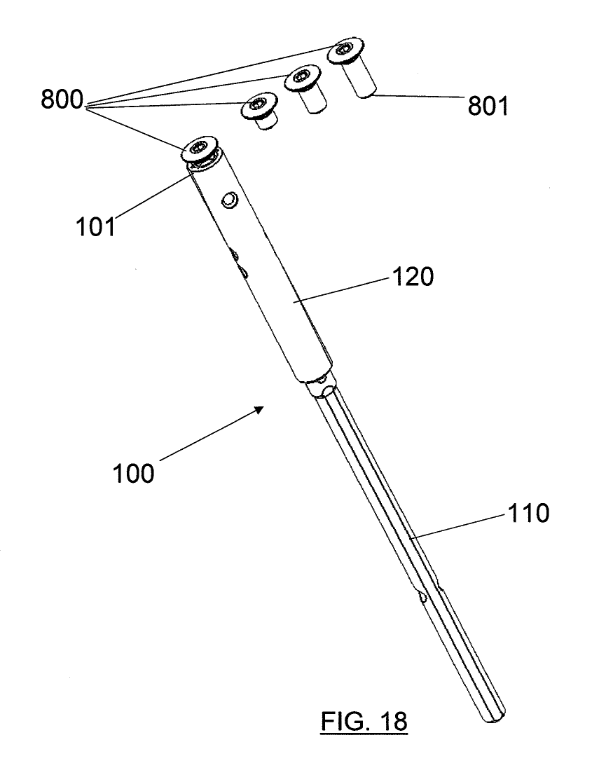

Referring now to FIG. 18, after the orthopedic device 100 has been compressed to the desired compression and the compression assembly 500 has been detached from the orthopedic device 100, a compression limiting end cap 800 may be installed in the proximal end 101 of the orthopedic device. The end cap 800 may include a threaded exterior for engagement with the threaded interior of the sleeve 120 to which the sleeve attachment bolt 560 was previously engaged. The end cap 800 may include a driver-receiving recess (such as a Torx® or Allen-key socket) to aid installation of the end cap 800. The end cap 800 may include a distal end 801 configured to engage the proximal end of the core 110 that is disposed within the sleeve 120 (and was previously attached to the core attachment bolt 520). By virtue of the distal end 801 of the end cap 800 engaging the proximal end of the core 110, the end cap 800 may prevent further compression of the core 110 into the sleeve 120.

Advantageously, the orthopedic device 100 may be configured for dynamic compression, wherein after the orthopedic device has been inserted, compressed, and the surgery is complete, the orthopedic device may further compress (by virtue of the core 110 sliding further into the sleeve 120 and the compression being held by the ratchet mechanism) during normal activity of the patient. Since the pawl member and ratchet surface cooperate to preclude movement of the core 110 out of the sleeve 120, as the orthopedic device 100 compresses, the orthopedic device 100 remains in its most compressed length. Such dynamic compression may be desirable to achieve better compression across the joint 720 as the bone portions 700, 701 fuse across the joint.

The amount of dynamic compression may be limited by insertion of the appropriate end cap 800. For example, after initial compression during the surgery and subsequent removal of the compression assembly, an end cap 800 may be installed in the proximal end 101 of the orthopedic device 100 such that the distal end 801 of the end cap 800 does not contact the proximal end of the core 110. The space between the distal end 801 of an inserted end cap 800 and the proximal end of the core 110 limits the maximum dynamic compression of the distance between the core 110 and the end cap 800. This maximum allowable dynamic compression may be varied based upon the selection of end caps 800 of different lengths. A longer end cap 800 will permit less (possibly zero) dynamic compression while a shorter end cap 800 will allow greater dynamic compression in the same patient. For example, for a patient in which the bone quality is poor (e.g., the bone is brittle or otherwise weakened), the maximum dynamic compression may be reduced such that the bone interface that is to be fused is not damaged by further fracture. Additionally, the threads used to engage the end cap 800 may also be used to facilitate removal of the orthopedic device from the patient by means of attaching a handle or other device to the threads (not shown).

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An orthopedic device for providing compression across a joint, fracture, or defect of a bone, comprising:
a core comprising;
at least one opening configured to receive a fastener for securing the core to a first bone portion, and
at least one of a ratchet or pawl member, and
a sleeve comprising;
at least one open end for slidably receiving the core,
at least one opening configured to receive a fastener for fastening the sleeve to a second bone portion, and
at least one of a ratchet or pawl member disposed at least partially within the sleeve and configured to engage at least one of the ratchet or pawl member of the core,
wherein the engaged ratchet and pawl member cooperate to allow the core to move into the sleeve in a direction through the open end and preclude the core from moving out of the sleeve in the opposite direction.

2. An orthopedic device according to claim 1, wherein the pawl member comprises at least one pawl surface that is biased against a ratchet surface of the ratchet.

3. The orthopedic device of claim 2, wherein the core comprises a slot, and wherein the slot is configured to receive at least a portion of the pawl member.

4. The orthopedic device of claim 2, wherein the sleeve comprises an inner bore into which the core is received, wherein the inner bore comprises the ratchet surface.

5. The orthopedic device of claim 1, further comprising an end cap configured to limit the distance the core can advance into the sleeve.

6. The orthopedic device of claim 1, further comprising at least one fastener insert disposed within the at least one opening of the sleeve, wherein the at least one fastener insert is configured to engage the fastener received through the at least one opening of the sleeve.

7. The orthopedic device of claim 6, wherein the core comprises at least one slot and wherein the at least one fastener insert is configured to pass through the at least one slot of the core and wherein the at least one fastener insert is configured to preclude relative rotation between the core and the sleeve.

8. An orthopedic device assembly for providing compression across a joint, fracture, or defect of a bone, comprising:
an orthopedic device comprising a core and a sleeve;
a target guide; and
a compression assembly configured to attach the orthopedic device to the target guide and to draw the core into the sleeve.

9. The orthopedic device assembly of claim 8, wherein the orthopedic device further comprises a pawl member disposed within the core and the sleeve, wherein the pawl member is configured to allow the core to be drawn into the sleeve and preclude the core from sliding out of the sleeve.

10. The orthopedic device assembly of claim 8, wherein the compression assembly comprises a core attachment bolt configured to engage the core and draw the core into the sleeve in response to the core attachment bolt turning with respect to the core.

11. The orthopedic device assembly of claim 8, wherein the orthopedic device further comprises at least one hole disposed in the core configured to receive a fastener and at least one hole disposed in the sleeve configured to receive a fastener.

12. The orthopedic device assembly of claim 11, wherein a fastener received in the at least one hole disposed in the core is configured to attach the core to a distal bone portion, and wherein a fastener received in the at least one hole disposed in the sleeve is configured to attach the sleeve to a proximal bone portion.

13. The orthopedic device assembly of claim 11, wherein the at least one hole disposed in the sleeve is configured to receive at least one fastener insert, wherein the fastener received through the at least one hole in the sleeve engages the at least one fastener insert.

14. The orthopedic device assembly of claim 13, wherein the core further comprises at least one slot and wherein the at least one fastener insert is configured to pass through the at least one slot of the core and preclude relative rotation between the sleeve and the core.

15. The orthopedic device assembly of claim 12, wherein compression is applied between the distal bone portion and the proximal bone portion in response to the compression assembly drawing the core into the sleeve.

16. A method of applying compression between a distal bone portion and a proximal bone portion using an orthopedic device, the method comprising:
inserting the orthopedic device through the proximal bone portion and into the distal bone portion, wherein the orthopedic device comprises a core and a sleeve;
fastening the core to the distal bone portion;
fastening the sleeve to the proximal bone portion; and
applying initial compression between the distal bone portion and the proximal bone portion.

17. The method of claim 16, further comprising maintaining compression between the distal bone portion and the proximal bone portion with a pawl member.

18. The method of claim 17, further comprising allowing for dynamic compression between the distal bone portion and the proximal bone portion after the initial compression has been achieved and maintaining the dynamic compression between the distal bone portion and the proximal bone portion with the pawl member.

19. The method of claim 18, further comprising limiting dynamic compression between the distal bone portion and the proximal bone portion.

20. The method of claim 16, wherein applying initial compression between the distal bone portion and the proximal bone portion comprises drawing the core into the sleeve.

21. The method of claim 20, wherein drawing the core into the sleeve comprises using a compression assembly to draw the core into the sleeve.

22. The method of claim 21, wherein using a compression assembly to draw the core into the sleeve comprises engaging a threaded bore of the core with a threaded stud and rotating the threaded stud relative to the core.

23. The method of claim 22, wherein the core is held in rotational alignment with the sleeve.
